SEQUENCE LISTING (5 sequences)

SEQ ID NO: 1 - RNA, 1460 nt, Aquabacterium sp.
  source 1..1460: mol_type = rRNA
cgctggcggcatgccttacacatgcaagtcgaacggtaacaggccgcaaggtgctgacgagtggcgaacgggtgagtaatgcatcggaacgtgcccagtagtgggggatagcccggcgaaagccggattaataccgcatacgacctgagggtgaaagggggggatcgcaagacctctcgctattggagcggccgatgtcagattagctagttggtggggtaaaggcctaccaaggcaacgatctgtagttggtctgagaggacgaccagccacactgggactgagacacggcccagactcctacgggaggcagcagtggggaattttggacaatgggcgcaagcctgatccagcaatgccgcgtgcaggaagaaggccttcgggttgtaaactgcttttgtcagggaagaaatcttctgggttaatacctcgggaggatgacggtacctgaagaataagcaccggctaactacgtgccagcagccgcggtaatacgtagggtgcgagcgttaatcggaattactgggcgtaaagcgtgcgcaggcggctttgcaagacagaggtgaaatccccgggctcaacctgggaactgcctttgtgactgcaaggctagagtacggcagagggggatggaattccgcgtgtagcagtgaaatgcgtagatatgcggaggaacaccaatggcgaaggcaatcccctgggcctgtactgacgctcatgcacgaaagcgtggggagcaaacaggattagataccctggtagtccacgccctaaacgatgtcaactggttgttggacggcttgctgttcagtaacgaagctaacgcgtgaagttgaccgcctggggagtacggccgcaaggttgaaactcaaaggaattgacggggacccgcacaagcggtggatgatgtggtttaattcgatgcaacgcgaaaaaccttacctacccttgacatgtcaagaatcttgcagagatgtgagagtgctcgaaagagaacttgaacacaggtgctgcatggccgtcgtcagctcgtgtcgtgagatgttgggttaagtcccgcaacgagcgcaacccttgtcatcagttgctacgaaagggcactctgatgagactgccggtgacaaaccggaggaaggtggggatgacgtcaggtcctcatggcccttatgggtagggctacacacgtcatacaatggccggtacagagggctgccaacccgcgagggggagccaatcccagaaaaccggtcgtagtccggatcgcagtctgcaactcgactgcgtgaagtcggaatcgctagtaatcgcggatcagcttgccgcggtgaatacgttcccgggtcttgtacacaccgcccgtcacaccatgggagcgggttctgccagaagtagttagcctaaccgcaaggagggcgattaccacggcagggttcgtgactggggtgaagtcataacaaggg

SEQ ID NO: 2 - DNA, 20 bp, synthetic construct
  source 1..20: mol_type = other DNA
agrgtttgatcmtggctcag

SEQ ID NO: 3 - DNA, 22 bp, synthetic construct
  source 1..22: mol_type = other DNA
tacggytaccttgttaygactt

SEQ ID NO: 4 - DNA, 20 bp, synthetic construct
  source 1..20: mol_type = other DNA
ccgtcaattcctttgagttt

SEQ ID NO: 5 - DNA, 20 bp, synthetic construct
  source 1..20: mol_type = other DNA
gtagcggtgaaatgcgtaga

(19) United States
(12) Patent Application Publication
(10) Pub. No.: US 2026/0286287 A1
(43) Pub. Date: Sep. 24, 2026

(54) NOVEL BACTERIAL STRAIN USEFUL FOR TREATING CUTANEOUS INFLAMMATION

(71) Applicants: PIERRE FABRE DERMO-COSMETIQUE, LAVAUR (FR); CENTRE NATIONAL DE LA RECHERCHE SCIENTIFIQUE (CNRS), PARIS (FR); SORBONNE UNIVERSITE, PARIS (FR)

(72) Inventors: Muriel BOURRAIN, CASTRES (FR); Audrey CALVEZ, CASTRES (FR); Martine MAITRE, CASTRES (FR); Katia RAVARD, CASTRES (FR); Philippe LEBARON, PALAU DEL VIDRE (FR)

(73) Assignees: PIERRE FABRE DERMO-COSMETIQUE, LAVAUR (FR); CENTRE NATIONAL DE LA RECHERCHE SCIENTIFIQUE (CNRS), PARIS (FR); SORBONNE UNIVERSITE, PARIS (FR)

(21) Appl. No.: 19/476,646

(22) PCT Filed: Apr. 18, 2024
(86) PCT No.: PCT/EP2024/060526

§ 371 Date: Oct. 17, 2025

(30) Foreign Application Priority Data
Apr. 18, 2023 (EP) 23305600.1

(51) Int. Cl. C12N 1/205; A61K 35/74; A61P 29/00; C12R 1/01

(52) U.S. Cl. CPC C12N 1/205; A61K 35/74; A61P 29/00; C12R 2001/01

(57) ABSTRACT

Provided herein is a novel bacterial strain isolated from groundwater, as well as bacterial extracts thereof which are useful for preventing and/or treating cutaneous inflammatory diseases, notably dermatological pathologies. Also provided are cosmetic or dermatological compositions containing such a bacterial extract as an active agent.

### FIELD OF THE DISCLOSURE

The present disclosure provides a novel bacterial strain isolated from groundwater. More particularly, the disclosure provides bacterial extracts which may be useful in the prevention and/or treatment of cutaneous inflammatory diseases, notably dermatological pathologies. The present disclosure also provides cosmetic or dermatological compositions containing such a bacterial extract as an active agent.

The novel bacterial strain isolated is a new mesophilic chemoheterotrophic, Gram-negative, oxidase-positive, and non-spore-forming rods bacterial strain belonging to the genus Aquabacterium that has been isolated from thermal spring water of deep aquifer in France. The present disclosure also discloses bacterial extracts obtained from such strain, their uses in the prevention and/or treatment of cutaneous inflammatory disorders as well as dermo-cosmetic compositions containing such extracts.

### BACKGROUND

Reactive skin is characterised by marked sensitivity to environmental or chemical factors (such as heat, cold, chemical, pollutants . . . ) and by the alteration of the skin barrier's impairing its ability to repair.

Advanced stage of reactive skin may lead to different cutaneous inflammatory diseases, such as atopic dermatitis. It is the cutaneous manifestation of atopy—i.e., chronic inflammatory dermatosis-occurring due to a genetically determined set of circumstances. Atopic dermatitis is one of the most common chronic diseases in the population. It is often accompanied by itching and pruritus, thus causing discomfort and annoyance in daily life (scratching, sleep loss, etc.).

Atopic dermatitis is a chronic inflammatory dermatological disease combining impairment of the skin barrier and skin inflammation. Notably, the skin barrier defect allows allergens to penetrate the upper layers of the epidermis. The allergens are then processed (internalised) by epidermal Langerhans cells and dermal dendritic cells. Langerhans cells are antigen-presenting cells that are capable of capturing skin antigens, preparing them and presenting them to T lymphocytes. This leads to activation of the Th2 (T helper 2) response, which results in the production of inflammatory cytokines such as IL-4 (Interleukin-4), IL-5 and IL-13 (see e.g., Bieber, Ann Dermatol. 2010, 22 (2): 125-137).

Accepted treatments include topical corticosteroids and immunomodulators, systemic agents whose frequent side effects limit long-term use, and emollients. The disease must therefore be treated over the long term. There is thus a need and a high demand for therapeutic alternatives for these inflammatory dermatoses.

The epidermis, the outermost layer of skin, provides a biological and physical barrier against invasion of the body by microorganisms and pathogens, damages from chemical products, loss of water and solutes. This physical barrier property of the epidermis is notably linked to its structure. The epidermis is conventionally divided into a basal layer of keratinocytes constituting the germinative layer of the epidermis, a so-called spinous layer consisting of several layers of polyhedral cells and finally, a set of upper layers called the corneal layer (or stratum corneum), consisting of keratinocytes in the terminal stage of their differentiation called corneocytes. The regenerative capacity of the epidermis is conferred by adult stem cells which allow regular replacement of the differentiated cells eliminated during keratinisation. This process is particularly crucial for barrier function maturation and maintenance.

Keratinocytes are crucial for the skin barrier function. Notably, the desmosomes connecting the keratinocytes of the spinous layer and the granular layer ensure the great cohesion and very high mechanical resistance of the tissue. In the keratinocytes of the granular layer, there are granules of keratohyaline which contain profilaggrin, and the lamellar bodies of Odland which are loaded with lipids. The watertightness of this “brick wall” is provided by an intercellular cement composed of specific lipids (cholesterol, cholesterol sulphate, free fatty acids and ceramides).

Corneocytes no longer have nuclei or organelles but are loaded with keratin filaments coated in filaggrin to form a fibrous matrix. In the most superficial layers, filaggrin is proteolyzed into amino acids and urocanic acid. The amino acids form the NMF (Natural Moisturizing Factor), whilst urocanic acid absorbs UV (Ultra Violet).

The association between the close cohesion of the keratinocytes of the spiny and granular layers, and the hydrophobic layer formed by the lipids and the corneocytes constitutes a real protective barrier for the skin. The stimulation of the proliferation and activity of keratinocytes triggers the thickening of the epidermis which reaches its optimal functionality: the skin barrier function, that prevents water loss.

There is still a need for efficient therapies of cutaneous inflammatory disorders, notably atopic dermatitis.

### SUMMARY OF THE DISCLOSURE

A first aspect of the present disclosure concerns a bacterial strain according deposited with the CNCM (Institut Pasteur, 75015 Paris, France) on Nov. 4, 2022, under the reference 1-5858.

A second aspect of the present disclosure concerns a method for preparing an extract of the disclosed bacterial strain comprising:
a) a bacterial culturing step comprising growing the bacterial strain in a suitable growth medium thereby obtaining a bacterial culture,
b) an extraction step comprising adding an organic solvent or a basic buffer to the bacterial culture of step a), thereby obtaining an extract of the bacterial strain.

A third aspect of the present disclosure concerns a bacterial extract obtainable according to the disclosed method.

A fourth object of the present disclosure concerns a composition comprising the disclosed bacterial extract optionally at least one cosmetically or dermatologically acceptable excipient.

Finally, a fifth aspect of the present disclosure concerns the use of the disclosed bacterial extract, the disclosed composition or the disclosed bacterial strain, as an active ingredient in the treatment and/or prevention of cutaneous inflammatory disorders.

### FIGURE LEGEND

FIG. 1: Neighbour-joining phylogenetic tree based on 16S rRNA gene sequences showing the relationships of strain LMB275T with the related Aquabacterium species. Filed circles at nodes indicated branches that were also recovered by using the maximum parsimony and the maximum likelihood algorithms. Bootstraps values (expressed as percentage of 1000 replications) over 70% are shown at the branching points. Bar 0.005 substitution per nucleotide position.

### DETAILED DESCRIPTION

The inventors have succeeded in isolating a strain, designated LMB275, belonging to a novel bacterial species from groundwater. This specific bacterial strain is particularly useful because it is capable of reducing cutaneous inflammation in atopic dermatitis. Indeed, the inventors have demonstrated that these bacterial extracts have a stimulatory effect on the differentiation of keratinocytes and an inhibitory effect on the cutaneous inflammation. This is notably illustrated by the reduction of inflammation gene expression caused by these bacterial extracts in an in vitro model of atopic dermatitis.

The bacterial strain has been further characterised by the inventors as a Gram-negative, oxidase-positive, and non-spore-forming rods bacterial strain belonging to the genus Aquabacterium. The species of the genus Aquabacterium are motile, rod-shaped, Gram-negative, oxidase-positive, catalase-negative bacteria which were first isolated from drinking water biofilms in Berlin. Identified species of the Aquabacterium genus include Aquabacterium citratiphilum, Aquabacterium parvum, Aquabacterium commune, Aquabacterium fontiphilum, Aquabacterium limnoticum, Aquabacterium olei, Aquabacterium tepidiphilum, Aquabacterium pictum, Aquabacterium lacunae, and Aquabacterium terrae.

However, DNA-DNA hybridisation relatedness between these species and our strain has shown that the present strain belongs to a new species of Aquabacterium.

Thus, in a first aspect, the present disclosure relates to a new bacterial strain of the genus Aquabacterium.

The strain LMB275 disclosed herein was deposited in accordance with the Budapest Treaty with the Collection Nationale de Cultures de Microorganismes (CNCM), Institut Pasteur, Paris, under the reference CNCM I-5858.

The present disclosure relates more specifically to a bacterial strain deposited with the CNCM (Institut Pasteur, Paris, France) on 4 Nov. 2022, under the reference I-5858.

Thus, one object of the disclosure is the bacterium deposited with the CNCM, under the reference CNCM I-5858, or a variant thereof.

As used herein, the term “variant” refers to a bacterial cell or a bacterial strain derived from a bacterial strain of interest, such as e.g., the present strain 1-5858. As used herein the term “derived from” means that the variant bacterial cell comprises substantially the same nucleotide sequence as the bacterial strain of interest, such as e.g., the present strain I-5858. For example, the variant bacterial cell disclosed herein may have up to 80%, 85%, 90%, 95%, 98% or 99% identity with the bacterial strain of interest, such as e.g., the present strain I-5858. A variant, as used herein, thus differs from the original bacterial strain of interest by at least one change in the genome. This change in the genome can be the result of e.g., a mutational event (natural or induced; random or site-directed). Such mutational events can result in e.g., one or more substitutions, insertions, and/or deletions. Alternatively, the change in the bacterial genome can be generated through recombination. A “variant” may have substantially the same functional activity as a reference bacterial strain, e.g. the present strain I-5858. For example, a variant of the present strain I-5858 is preferably still capable of stimulating keratinocytes differentiation and inhibiting cutaneous inflammation.

The present bacterial strain has been characterised as a novel mesophilic chemoheterotrophic bacterial strain that grows under aerobic/microaerophilic conditions, and was isolated from the thermal spring water of a deep aquifer in France. Cells were oxidase-positive and catalase-negative, with a polar flagellum. Phylogenetic analyses based on 16S rRNA gene sequences indicated that strain LMB275 formed a phylogenetic lineage in the genus Aquabacterium, and was found to be most closely related to Aquabacterium parvum B6T (99.1%), Aquabacterium commune B8T (97.9%) and Aquabacterium citratiphilum B4T (97.0%). The DNA-DNA relatedness between strain LMB275 and A. parvum was below 70.0%. The DNA G+C content was 67 mol %. Strain LMB275 was isolated from thermal spring water collected at the catchment point of a deep aquifer (180 meters deep) located in the north of the Hérault department in France at the eastern end of the “Montagne Noire” mountain. More specifically, this aerobic and microaerophile bacteria LMB275 is rod-shaped with a length of about 2.6±0.2 μm and a width of about 0.6±0.1 μm. This bacterium is motile using a single polar flagellum.

The gene coding for 16S rRNA was almost completely sequenced (1460 bp). This sequence corresponds to sequence SEQ ID NO:1.

The present disclosure also encompasses variants of the present bacterial strain which comprise a 16S rRNA gene comprising sequence SEQ ID NO: 1, or any sequence having at least 80% identity with sequence SEQ ID NO: 1, advantageously at least 85%, at least 90%, at least 95%, or at least 97% and more preferentially at least 98% identity with sequence SEQ ID NO: 1. Other characteristics of said bacterium LMB275 will be detailed below in the examples.

As used herein, “percentage identity” between two nucleic acid sequences refers to a percentage of identical nucleotides between the two sequences to be compared, obtained after the best alignment (optimal alignment), this percentage being purely statistical and the differences between the two sequences being distributed at random and over their entire length. Sequence comparisons between two nucleic acid sequences are traditionally carried out by comparing these sequences after they have been optimally aligned, which can be done by segment or by “comparison window”. Optimal alignment of the sequences for comparison can be achieved, in addition to manually, by means of the local homology algorithm of Smith and Waterman (1981), by means of the local homology algorithm of Needleman and Wunsch (1970), using the Pearson and Lipman (1988) similarity search method, using computer software using these algorithms: GAP, BESTFIT, FASTA and TFASTA in the Wisconsin Genetics Software Package, Genetics Computer Group, 575 Science Dr., Madison, Wis., or by the BLAST (Basic Local Alignment Search Tool) N or BLAST P comparison software.

The percentage identity between two nucleic sequences is determined by comparing these two optimally aligned sequences in which the nucleic acid sequence to be compared may include additions or deletions with respect to the reference sequence for optimal alignment between these two sequences. The percentage identity is calculated by determining the number of identical positions for which the nucleotide is identical between the two sequences, dividing this number of identical positions by the total number of positions in the comparison window and multiplying the result by 100 to obtain the percentage identity between these two sequences.

For example, the BLAST program can be used, specifically “BLAST 2 sequences” (Tatusova et al, 1999) on the available website http://www.ncbi.nlm.nih.gov/gorf/b12.html, the default parameters are used (in particular for the parameters “open gap penalty”: 5, and “extension gap penalty”: 2; the matrix chosen is for example the matrix “BLOSUM 62” suggested by the program). The percentage identity between the two sequences to be compared is calculated directly by the program. It is also possible to use other programs such as “ALIGN” or “Megalign” (DNASTAR).

A further object of the present disclosure relates to extracts of the bacterial strain belonging to the genus Aquabacterium deposited under CNCM number 1-5858. These extracts can be used as active ingredient in the treatment and/or prevention of cutaneous inflammatory disorders, notably dermatological pathologies.

In particular, the bacterial extract disclosed herein can be obtained by a method comprising the steps of:
a) a bacterial culturing step comprising growing the bacterial strain in a suitable growth medium thereby obtaining a bacterial culture,
b) an extraction step comprising adding an organic solvent or a basic buffer to the bacterial culture of step a),
   thereby obtaining an extract of the bacterial strain. The term “bacterial extract” refers to any extract or fraction of a bacterial cell, preferably a cell of strain 1-5858, or any active fraction of said extract. In particular, a bacterial extract can comprise the biomolecules produced and secreted in the medium by the bacterial cell, as well as intercellular components and components from the membrane.

The term “biomolecules” refers to biomolecules secreted into the growth medium and solubilised in this medium and also to biomolecules produced by the bacterium and not secreted into the growth medium. Such biomolecules may comprise intracellular components, components arising from the membrane, from the periplasmic space and/or from the extracellular space, such as the proteins, peptides, primary and/or secondary metabolites, genetic materials. More particularly, these biomolecules consist mainly of the proteins, peptides and secondary metabolites produced by the bacterium and intracellular components.

The expression “bacterial culture” refers to the culture of a bacterium in a growth medium, allowing the growth and recovery of this bacterium.

The expression “growth medium” refers to any medium containing at least the nutrients necessary for bacterial growth and multiplication. Bacteria can be grown in liquid, solid or semi-liquid media. Preferably, the growth medium is a liquid medium allowing the growth and recovery of bacterium and allowing the recovery of biomolecules secreted by said bacterium. Preferably, the growth medium is an aqueous growth medium.

A suitable growth medium contains nutrients which promote bacterial growth and multiplication. Generally, a suitable growth medium comprises water, a carbon source, a nitrogen source, and appropriate salts. By way of illustration, a particular exemplary growth medium is described below in the examples.

Preferably, the bacterial extract disclosed herein, can be obtained by growing the present bacterial strain in a medium allowing the growth, development, and multiplication of said bacterial strain and the recovery of the bacterial biomolecules.

In a first instance, the bacterial extract of bacterium LMB275 can be obtained by a method comprising:
a) a bacterial culturing step comprising growing the bacterial strain in a suitable growth medium thereby obtaining a bacterial culture,
b) an extraction step comprising adding an organic solvent or a basic buffer to the bacterial culture of step a),
   thereby obtaining an extract of the bacterial strain. By “basic buffer”, it is herein referred to any buffer having a pH comprised between 8 and 12, preferably between 9 and 11, and more preferably between 9.5 and 10.5. Basic buffers comprise such buffers as e.g., a Tris buffer and an arginine buffer. Preferably the basic buffer is an arginine buffer. The arginine concentration is preferably comprised between 0.1 and 1 M, more preferably between 0.3 and 0.5 M.

Preferably, the extraction step b) comprises the further steps of:
b1) separating the liquid and solid phases,
b2) adding the basic buffer to the liquid phase, and
b3) recovering the buffered liquid phase.

The bacterial extract S0 thus recovered comprises biomolecules secreted by the strain in the supernatant.

Liquid/solid phase separation can be carried out by any technique known to the skilled person such as e.g., centrifugation, sedimentation, filtration, ultrafiltration, precipitation, etc, or any combination of techniques known to the skilled person. It is in particular advantageous to centrifuge the bacterial culture, so that the bacterial biomass pellet is separated from the medium supernatant. In a particular instance, the bacterial culture is centrifuged at a speed between 5000 and 14000 rpm (round per minute), preferably between 7500 and 12500 rpm, more preferably between 9500 and 11000 rpm. In another particular instance, the bacterial culture is centrifuged between 1 and 60 minutes, preferably between 2 and 45 minutes, more preferably between 4 and 30 minutes, even more preferably between 5 and 20 minutes. In yet another particular instance, the bacterial culture is centrifuged at a temperature comprised between +1° C. and +20° C., preferably between +2° C. and +12° C., more preferably, between +4° C. and +6° C.

In a specific instance, the basic buffer is added to the liquid phase and the mixture is incubated for 1 to 7 hours, preferably for 2 to 6 hours, more preferably for 3 to 5 hours. In another instance, the basic buffer is added to the liquid phase and the mixture is incubated between +1° C. and +7° C., preferably between +2° C. and +5° C., more preferably between +3° C. and +4° C.

Preferably, the bacterial extract S0 can be obtained by growing the bacterial strain LMB275 in a suitable growth medium, separating the liquid and solid phases of the bacterial culture, for example by centrifugation, adding the basic buffer to the liquid phase and recovering the buffered liquid phase. The liquid/solid separation in this method allows to separate the solid phase, comprising biomass pellet, and cell debris, from the liquid phase, which comprises the soluble molecules of the bacterial secretome.

Generally, the term “secretome” is used to describe all the biomolecules secreted and excreted into the culture medium and solubilised in this medium, by a cell, a tissue or an organism. In the present case, secretome must be understood to refer to all the biomolecules secreted and externalised by bacterium LMB275, and this with no modification, deterioration or lysis of the bacterial cell. More particularly, these biomolecules consist mainly of the proteins, peptides and secondary metabolites secreted and externalised by the bacteria into the culture medium. The expression “secondary metabolites” is used herein to refer to the small molecules that the present bacteria, in particular LMB275, produce, secrete and excrete into the culture medium. By way of illustration, such secondary metabolites may be soluble molecules such as bacteriocins, non-ribosomal peptides, siderophores, lipopeptides or polyketides, or molecules such as terpenes, pyrazines, indoles, or sulphide derivatives.

It is also possible to separate the growth medium from the bacterial biomass after adding the basic buffer, then obtaining an extract ES0.

Accordingly, the extraction step b) comprises the further steps of:
b1′) adding the basic buffer to the bacterial culture,
b2′) separating the liquid and solid phases, and
b3′) recovering the buffered liquid phase.

The bacterial extract ES0 thus recovered comprises biomolecules secreted by the strain in the supernatant and periplasmic biomolecules.

In a specific instance, the basic buffer is added to the bacterial culture and the mixture is incubated for 1 to 7 hours, preferably for 2 to 6 hours, more preferably for 3 to 5 hours. In another specific instance, the basic buffer is added to the bacterial culture and the mixture is incubated between +1° C. and +7° C., preferably between +2° C. and +5° C., more preferably between +3° C. and +4° C.

The separation of the liquid and solid phases can be carried out by any technique known to the skilled person such as e.g., centrifugation, sedimentation, filtration, ultrafiltration, precipitation, etc, or any combination of techniques known to the skilled person. Advantageously, the liquid and solid phases of the buffered bacterial culture are separated by centrifugation. In a particular instance, the buffered bacterial culture is centrifuged at a speed between 5000 and 14000 rpm (round per minute), preferably between 7500 and 12500 rpm, more preferably between 9000 and 11000 rpm. In another particular instance, the buffered bacterial culture is centrifuged between 1 and 60 minutes, preferably between 2 and 45 minutes, more preferably between 4 and 30 minutes, even more preferably between 5 and 20 minutes. In yet another particular instance, the buffered bacterial culture is centrifuged at a temperature comprised between +1° C. and +20° C., preferably between +2° C. and +12° C., more preferably, between +4° C. and +6° C.

Preferably, the bacterial extract ES0 can be obtained by growing the bacterial strain LMB275 in a suitable growth medium, adding a basic buffer to the bacterial culture, separating liquid and solid phases of the buffered bacterial culture, for example by centrifugation, and recovering the liquid phase. The liquid/solid separation in this method allows to separate the solid phase, comprising biomass pellet, and cell debris, from the liquid phase, comprising the soluble molecules secreted during growing phase and biomolecules from the periplasm.

Advantageously, the method disclosed in the above instances further comprises a step c) of filtrating the buffered liquid phase of step b3) or b3′). Accordingly, one or more filtration steps may be performed to clarify the extract.

Filtration may be carried out by any appropriate means allowing clarification of the liquid phase, or the buffered liquid phase. Such clarification by filtration allows the removal of suspended particles that would not have been removed in the second liquid/solid separation step and aims at producing a purified, clear bacterial extract according to the disclosure.

Filtration can be carried out by any means of filtration, ultrafiltration or diafiltration.

Advantageously, filtration is carried out on a filter or filter cartridge having a cut-off of 0.4 μm, preferably 0.2 μm. In this case, the bacterial extract is characterised in that the compounds present in the bacterial extract have a size which is less than or equal to 0.2 μm.

Preferably, electrostatically uncharged filters or prefilters may be used to avoid any absorption of the biomolecules responsible for all or part of the activity of the extract.

Alternatively, step b) of the method disclosed herein comprises adding an organic solvent, rather than a basic buffer, to the bacterial culture of step a). It will be easily realised that the nature of the extracts will be affected in consequence. Whereas the extracts S0 and ES0 described above contain proteins (see e.g., the examples), extracts L0 and BL0 obtained after organic solvent addition only contain molecules which are soluble in the organic solvent used.

Advantageously, the extraction step b) comprises the further steps of:
b1″) adding the organic solvent to the bacterial culture,
b2″) separating the aqueous and organic phases, and
b3″) recovering the organic phase, and optionally drying the organic phase.

By “organic solvent”, it is herein referred to carbon-based solvents capable of dissolving other substance. In particular, “organic solvent” refers to an agent that is hydrophobic or lipophilic, and is not a lipid. As used herein, “hydrophobic” refers to an agent that is repelled from a mass of water. As used herein, “lipophilic” refers to an agent that dissolves lipids. Non-limiting examples of organic solvents include substituted and unsubstituted C₄-C₈ alkyls (e.g., hexane and the like), C₅-C₁₂ cylcolalkyls, C₄-C₁₂ alkenes, C₁-C₈ alcohols (e.g., butanol, iso-propanol and the like), C₁-C₈ aldehydes, C₄-C₈ ethers, C₁-C₈ esters, C₆-C₁₂ aryls, C₁-C₈ amides, C₅-C₁₂ heteroaryls, and combinations thereof. Preferably, the organic solvent used in the methods disclosed herein is an alcohol in C₁-C₈ or C₁-C₈ esters, e.g., an alkyl acetate. In a preferred instance, the C₁-C₈ alcohol is butanol. In another preferred instance, the C₁-C₈ ester is ethyl acetate.

In a specific instance, the organic solvent is added to the bacterial culture and the mixture is incubated for 1 to 7 hours, preferably for 2 to 6 hours, more preferably for 3 to 5 hours. In another specific instance, the organic solvent is added to the bacterial culture and the mixture is incubated between +1° C. and +7° C., preferably between +2° C. and +5° C., more preferably between +3° C. and +4° C.

The liquid/liquid separation can be carried out by any technique known to the skilled person such as e.g., centrifugation, sedimentation, etc, or any combination of techniques known to the skilled person. Advantageously, the organic and aqueous phases of the bacterial culture after addition of the organic solvent are separated by centrifugation. In a particular instance, the bacterial culture after addition of the organic solvent is centrifuged at a speed between 5000 and 14000 rpm (round per minute), preferably between 7500 and 12500 rpm, more preferably between 9000 and 11000 rpm. In another particular instance, the bacterial culture after addition of the organic solvent is centrifuged between 1 and 60 minutes, preferably between 2 and 45 minutes, more preferably between 4 and 30 minutes, even more preferably between 5 and 20 minutes. In yet another particular instance, the bacterial culture after addition of the organic solvent is centrifuged at a temperature comprised between +1° C. and +20° C., preferably between +2° C. and +12° C., more preferably, between +4° C. and +6° C.

The organic phase is optionally dried. Any method known to the skilled person can be used for drying the organic phase. For example, the organic phase can be dried by evaporation, using a rotary evaporator.

Preferably, the bacterial extracts BL0 or L0 can be obtained by growing the bacterial strain in a suitable growth medium, adding an organic solvent to the bacterial culture, separating the aqueous and organic phases of the bacterial culture, and recovering the organic phase. Optionally, the organic phase can be dried, for example using a rotary evaporator to evaporate the liquid.

The different steps will be described in more detail in the examples. It must be understood that any modification of the process, media or sequence of steps that seems obvious to a skilled person with regard to the present description must be considered as falling within the scope of the present disclosure.

In another aspect, the present disclosure provides a bacterial extract obtained, or obtainable, by a method described above.

In another aspect, the disclosure provides a composition comprising the bacterial strain or the bacterial extract described herein. Preferably, the composition is suitable for topical application. The composition, the bacterial extract or the bacterial strain according to the disclosure may be used as an active ingredient in the treatment and/or prevention of cutaneous inflammatory disorders in a subject.

The term “subject” is used here to refer to any human being, whether an adult or a child. A “child” as used herein refers to an individual whose age is less than or equal to 16 years. An “adult” as used herein is a person who is not a child, i.e., a person over the age of 16.

The present disclosure provides a solution to the prevention and/or treatment of these cutaneous inflammatory disorders by the isolation, the characterisation and the fractionation of a never-before-described bacterium.

In an instance, the disclosure provides a composition comprising the bacterial strain according to the disclosure, or an extract thereof and optionally at least one cosmetically or dermatologically acceptable excipient.

Preferably, the bacterial extract comprised in the composition is obtained, or obtainable, by a method described above.

Preferably, the composition is a cosmetic or pharmaceutical composition.

The composition may further comprise at least one cosmetically or dermatologically acceptable excipient.

As used herein, “cosmetically or dermatologically acceptable” means that which is useful in the preparation of a cosmetic or dermatological composition which is generally safe, nontoxic and neither biologically nor otherwise undesirable and which is acceptable for cosmetic or dermatological use, notably by topical application.

According to a particular instance, the composition described herein is in a form suitable for topical application.

The present cosmetic or dermatological compositions may be in the forms that are generally known for topical administration, i.e., lotions, foams, gels, dispersions, emulsions, sprays, serums, masks or creams, jellies, in particular micellar jellies, with excipients allowing in particular skin penetration in order to improve the properties and accessibility of the active principle. Advantageously, the cosmetic or dermatological composition will be a cream, a rich cream, a lotion, an eye care product, a UV care product.

These compositions generally contain, in addition to the compounds of the bacterial extract disclosed herein, a physiologically acceptable medium, generally based on water or solvent, for example alcohols, ethers or glycols. They may also contain surfactants, complexing agents, preservatives, stabilisers, emulsifiers, thickeners, gelling agents, humectants, emollients, trace elements, essential oils, fragrances, dyes, matting agents, chemical or mineral filters, moisturisers, thermal waters, etc.

Such compositions can be manufactured according to processes well-known to the skilled person.

In a further aspect, the disclosure provides a bacterial strain, a bacterial extract or a composition disclosed herein, for use as an active ingredient in the treatment and/or prevention of cutaneous inflammatory disorders.

In another aspect, the present disclosure provides the use of the bacterial strain, the bacterial extract or the composition disclosed herein for the preparation of manufacture of a medicinal product intended to treat and/or prevent cutaneous inflammatory disorders.

In yet another aspect, the present disclosure provides a method for treating and/or preventing cutaneous inflammatory disorders, preferably dermatological pathologies in a subject in need thereof, comprising the administration of the bacterial strain, the bacterial extract or the composition disclosed herein.

The disclosure also relates to a method for preventing and/or treating cutaneous inflammatory disorders comprising administering to a subject in need thereof an effective amount of a bacterial strain, a bacterial extract or composition disclosed herein.

As used herein, the term “cutaneous inflammatory disorders” is used to describe any skin condition caused by chronic inflammation or for which an acute or chronic inflammation is a recurrent symptom. These conditions affect both the skin itself (i.e., the epidermis, dermis and/or hypodermis), as well as the pores of the skin, the attached sweat and sebaceous glands, and/or the microbiote.

Inflammation is a normal immune defence reaction of the body to an insult of the following types: infectious, thermal, mechanical, chemical, lesional or allergic. It is characterised by four points: redness, heat, swelling and pain. Acute skin inflammation is an immediate response to a harmful agent, of short duration (a few days or weeks), often with a sudden onset and characterised by intense swelling. Acute inflammations heal spontaneously or with treatment. Chronicity occurs when the inflammation does not heal spontaneously, persists or worsens for several months or even years. The inflammatory reaction is a dynamic process involving several successive steps: vascular (vasodilation), leukocyte extravasation and immune cell chemotaxis and finally cleansing. Leukocyte extravasation refers to the active crossing of vascular walls and the accumulation of circulating immune cells, lymphocytes, neutrophils and monocytes in the lesion site. Neutrophils have the function of attracting other inflammatory cells by chemotaxis and of cleaning the injured site by secreting antimicrobial substances and proteases. Monocytes migrate by chemotaxis and differentiate into macrophages which clean the damaged area. They secrete growth factors, inflammatory cytokines, such as IL-1, notably IL-1β, TNFα (Tumour Necrosis Factor), proteases, prostaglandins and IFNs (Interferons) allowing the maintenance and/or amplification of inflammation. Cleansing follows the vascular phase and is contemporary with leukocyte extravasation. This is a process in which necrotic tissue and pathogens are removed.

Inflammatory dermatoses are affections of the skin and mucous membranes, often painful, which are characterised by unsightly manifestations such as redness and scaly patches. Several pathologies are grouped under the name of inflammatory dermatoses, including atopic dermatitis, psoriasis, rosacea, lichen planus, pruritis, seborrheic dermatitis, and acne.

Preferably, the cutaneous inflammatory disorders treated and/or prevented by the disclosure include inflammatory dermatosis such as atopic dermatitis, pruritis, psoriasis, rosacea, lichen planus, seborrheic dermatitis or acne.

Pruritus can be observed in many cutaneous dermatoses, i.e., pruriginous dermatoses such as atopic dermatitis, contact dermatoses, psoriasis, lichen planus, and ectoparasitoses and insect bites. Pruritus is defined as an unpleasant sensation that causes the need to scratch.

Atopic dermatitis, also known as eczema, is the cutaneous manifestation of atopy. It is a chronic inflammatory dermatosis, occurring on a genetically determined terrain. It affects 15 to 30% of children and 2 to 10% of adults. Its prevalence is constantly increasing in industrialised countries, it has doubled or even tripled over the last three decades and is now considered a major public health concern. Atopic dermatitis is often associated with other atopic disorders, such as allergic rhinitis and asthma. The condition most often appears in early childhood and is characterised by repeated rashes over several years. It evolves in flare-ups interspersed with spontaneous remissions. The lesions are characterised by significant skin dryness associated with inflammatory manifestations: erythematous, papular, vesicular, scaly and very pruritic eruptions. Barrier function markers, as well as inflammation and immunity markers, are marker of atopic dermatitis (Tokura et al., 2022).

Acne vulgaris is a chronic inflammatory disease of the pilosebaceous units. The pathogenesis of acne involves an interplay of four main factors: Dysseborrhea, follicular hyperkeratosis, cutaneous microbiome dysbiosis and local immuno-inflammation (Dagnelie et al. 2019).

Psoriasis is also a cutaneous inflammatory disease with a chronic progression; it affects 2% of the population. Along with atopic dermatitis, psoriasis is one of the most common chronic cutaneous inflammatory diseases. It is characterised by abnormal growth of epidermal cells associated with an inflammatory reaction (Raharja et al., 2021). The central mechanism of the inflammation phenomenon is related to the action of the immune system T cells, predominantly Th1 cells, which initiate and maintain the inflammatory process and stimulate the excessive proliferation of keratinocytes which then proceed through an accelerated and incomplete differentiation phase. Keratinocytes express receptors which make them sensitive to inflammatory signals and release pro-inflammatory mediators. Psoriatic inflammation is thus maintained by mutual stimulation of T cells and keratinocytes.

Rosacea is a chronic cutaneous inflammatory disease mainly affecting the cheeks, nose, chin, and forehead. Rosacea is characterised by recurrent episodes of flushing or transient erythema, persistent erythema, phymatous changes, papules, pustules, and telangiectasia. (Van Zuuren et al. 20211). The innate immunity is triggered by Rosacea and lead to upregulation of keratinocyte-derived toll-like receptor 2 (TLR2) and proteinase-activated receptor 2 (PAR2). These promote expression of the antimicrobial peptide cathelicidin, which is subsequently activated to bioactive LL-37 by kallikrein 5 (KLK-5) protease, leading to erythema and angiogenesis. TLR2 activation can elicit pustule formation, erythema, telangiectasia, and inflammation via expression of cytokines, chemokines, proteases, and angiogenic factors. AR2 activation leads to inflammation, pruritus, and pain combined with recruitment of T lymphocytes and neutrophils.

Lichen planus (LP) is an inflammatory mucocutaneous condition, affecting the mucous membrane, with characteristic violaceous polygonal flat-topped papules and plaques. Pruritus is often severe (Katta et al. 2000). LP is estimated to affect 0.5% to 2.0% of the general population. An early event in the disease mechanism involves keratinocyte antigen expression or unmasking of an antigen that may be a self-peptide or a heat shock protein, followed by T cells migration (Gupta et al. 2015).

Seborrheic dermatitis (SD) is a common skin condition characterised by scaling, erythema, and itching occurring most often on the scalp, face, chest, back, axilla, and groin (Clark et al. 2015). SD prevalence is 1% to 3% in the general population and 34% to 83% in immunocompromised persons. The skin changes are thought to result from an inflammatory response to a common skin organism, Malassezia yeast. Malassezia is a normal component of skin flora, but in persons with seborrheic dermatitis, the yeast invades the stratum corneum, releasing lipases that result in free fatty acid formation and trigger inflammation. The inflammation causes stratum corneum hyperproliferation (scaling) and incomplete corneocyte differentiation, which alters the stratum corneum barrier and impairs its function, thus increasing access for Malassezia and allowing water to more readily leave the cells.

In one instance of the present disclosure, the bacterial strain, the bacterial extract and the composition disclosed herein can modulate the expression of at least one biological marker.

Accordingly, the disclosure relates to a bacterial strain, a bacterial extract, or a composition disclosed herein, for use as an active ingredient in the treatment and/or prevention of cutaneous inflammatory disorders, wherein the use comprises the modulation of the expression of at least one biological marker.

It will be understood that the disclosure also relates to the use of the bacterial strain, the bacterial extract and the composition disclosed herein for the preparation of manufacture of a medicinal product intended to treat and/or prevent cutaneous inflammatory disorders, wherein the treatment or prevention comprises the modulation of the expression of at least one biological marker.

In yet another instance, the present disclosure provides a method for treating and/or preventing cutaneous inflammatory disorders, preferably dermatological pathologies in a subject in need thereof, comprising the administration of the bacterial strain, the bacterial extract, or the composition disclosed herein and the modulation of the expression of at least one biological marker.

The disclosure also relates to a method for preventing and/or treating cutaneous inflammatory disorders comprising administering to a subject in need thereof an effective amount of a bacterial strain, a bacterial extract, or composition disclosed herein and modulating the expression of at least one biological marker.

This modulation may correspond, depending on the case, and in particular depending on the nature of the biological marker, to an increase or a decrease in the expression of said marker. Similarly, the bacterial strain, the bacterial extract or the composition disclosed herein is capable of preventing and/or treating at least one cutaneous inflammatory disorder, if the expression of at least one biological marker is modulated further to the administration of the bacterial strain, bacterial extract or composition. As used herein, the terms “prevent”, “prevention” and “preventing” refer to the reduction in the risk of acquiring or developing a given condition, e.g., a cutaneous inflammatory disease, or the reduction or inhibition of the recurrence or said condition in a subject who is not ill, but who has been or may be near a subject with the disease. It is also to be appreciated that the various modes of treatment or prevention of medical conditions as described are intended to mean “substantial,” which includes total but also less than total treatment or prevention, and wherein some biologically or medically relevant result is achieved. The terms “treating” or “treatment” refer to administering or the administration of the bacterial strain, bacterial extract or composition disclosed herein described herein in an amount, manner, and/or mode effective to improve a condition, symptom, or parameter associated with a disorder, e.g., a cutaneous inflammatory disease, or to prevent progression or exacerbation of the disorder (including secondary damage caused by the disorder) to either a statistically significant degree or to a degree detectable to one skilled in the art. When a disease, e.g., a cutaneous inflammatory disease, or a symptom thereof, is being treated, administration of the substance (i.e., the bacterial strain, bacterial extract or composition in the present case) typically occurs after the onset of the disease or symptoms thereof. When a disease, e.g., a cutaneous inflammatory disease, or symptoms thereof, are being prevented, administration of the substance (i.e., the bacterial strain, bacterial extract or composition in the present case) typically occurs before the onset of the disease or symptoms thereof.

Modulation can be either an increase or a decrease. In one instance, the expression of a biological marker is modulated means that the expression of the marker is increased. In another instance, the expression of a biological marker is modulated means that the expression of the marker is decreased.

The term “increased”, as used here regarding the expression of a marker, means that the expression of such marker is more important when a bacterial strain, a bacterial extract, or composition disclosed herein is administered to the skin, than when nothing is administered to the skin. The expression of such marker when nothing is administered to the skin is also named the original quantity.

Particularly, the quantity of marker's expression is greater, for example, a quantity slightly greater than the original quantity, or for example, a quantity in great excess of the original quantity, including all quantities in between. Alternatively, “increase” may refer to a quantity or activity that is at least 1%, 2%, 3%, 4%, 5%, 6%, 7%, 8%, 9%, 10%, 11%, 12%, 13%, 14%, 15%, 16%, 17%, 18%, 19%, or 20% more than the quantity or activity for which the increased quantity or activity is being compared, or at least 30%, 40%, 50%, 60%, 70%, 80%, 90%, 100%, 110%, 120%, 130%, 140%, 150%, 160%, 170%, 180%, 190%, 200%, 250%, 300%, 350%, 400%, 450%, 500%, 550%, 600%, 650%, 700%, 750%, 800%, 900%, 950%, 1 000%, 1 100%, 1,200%, 1,300%, 1,400%, 1,500%, 1,600%, 1,700%, 1,800%, 1,900%, 2,000%, 2,200%, 2,400%, 2,600%, 2,800%, 3,000%, 3,200%, 3,400%, 3,600%, 3,800% or 4,000% more than the quantity or activity for which the increased quantity or activity is being compared. The terms “increased”, “greater than”, “more than” and “increased” are used interchangeably here.

The term “decreased”, as used here regarding the expression of a marker, means that the expression of such marker is less important when a bacterial strain, a bacterial extract, or composition disclosed herein is administered to the skin of the subject than when nothing is administered to the skin, or to a skin model. The expression of such marker when nothing is administered to the skin is also named the original quantity.

Particularly, the quantity of marker's expression is smaller, for example, a quantity slightly less than the original quantity, or e.g., a quantity greatly reduced from the original quantity, including all quantities in between. Alternatively, “decrease” may refer to a quantity or activity that is at least 1%, 2%, 3%, 4%, 5%, 6%, 7%, 8%, 9%, 10%, 11%, 12%, 13%, 14%, 15%, 16%, 17%, 18%, 19%, or 20% less than the quantity or activity for which the decreased quantity or activity is being compared, or at least 30%, 40%, 50%, 60%, 70%, 80%, 90%, 100%, 110%, 120%, 130%, 140%, 150%, 160%, 170%, 180%, 190%, 200%, 250%, 300%, 350%, 400%, 450%, 500%, 550%, 600%, 650%, 700%, 750%, 800%, 900%, 950%, 1 000%, 1 100%, 1 200%, 1 300%, 1 400%, 1 500%, 1 600%, 1 700%, 1 800%, 1,900%, 2,000%, 2,200%, 2,400%, 2,600%, 2,800%, 3,000%, 3,200%, 3,400%, 3,600%, 3,800% or 4,000% less than the quantity or activity for which the decreased quantity or activity is being compared. The terms “decreased”, “less than”, and “reduced” are used interchangeably here.

The term “biomarker” used herein refers to a characteristic that is objectively measured and evaluated as an indicator of normal biological processes, pathogenic processes, or pharmacological responses to a therapeutic intervention. A biomarker therefore refers to a range of different substances and parameters. For example, a biomarker may be a substance whose detection indicates a particular disease state (e.g., the presence of C-reactive protein as a marker of infection), or conversely a substance whose detection indicates a specific physiological state. Preferably, said biomarker is a biomarker of a cutaneous inflammatory disorder if its level of expression is different in healthy skin and in skin exhibiting the clinical features of said cutaneous inflammatory disorder.

For the purposes of the present disclosure, the term “biological marker” is understood to mean a characteristic which is objectively measured and evaluated as an indicator of normal biological processes, pathogenic processes or pharmacological responses to a therapeutic intervention. A biological marker therefore designates a whole range of various substances, activities and parameters.

The biological marker disclosed herein is preferably a gene, a product of a gene, such as its transcripts and the peptides generated by translation from its transcripts, a lipid, a sugar or a metabolite. More preferably, the biological marker disclosed herein is a gene or any of the products of a gene such as its transcripts and the peptides obtained from its transcripts.

The biological marker is therefore advantageously a marker selected from the group consisting of barrier function markers, differentiation markers, lipid markers, desquamation markers, antimicrobial markers, innate immunity markers and inflammation markers.

The person skilled in the art seeking to determine to which class a biomarker belongs can easily consult the relevant scientific literature or refer to public databases such as, for example, those gathered on the website of the National Center for Biotechnology Information (http://www.ncbi.nlm.nih.gov/guide/).

The term “barrier function marker” as used herein refers to a marker specifically expressed in the outermost layers of the epidermis and which participate in barrier function, and preferably in the stratum corneum. Differentiation marker and lipid markers are members of the group of barrier function marker.

The term “differentiation marker” as used herein refers to a marker involved in skin cell differentiation, preferably in keratinocyte differentiation. Preferably, the differentiation marker is selected from the group consisting of Small Proline Rich Protein 1B (SPRR1B), sciellin (SCEL), involucrin (IVL), and caspase 14 (CASP14).

The term “lipid marker” as used herein refers to a marker involved in lipid metabolism, preferably lipid metabolism in the skin. The term “lipid” is understood here to mean any liposoluble (i.e., lipophilic) natural molecule. Lipids are a heterogeneous group of compounds possessing numerous essential biological functions. Preferably, the lipid marker is a member of the cytosolic sulfotransferase family, more preferably, the sulfotransferase 2B1 member (SULT2B1).

The term “desquamation” is understood here to mean the loss of superficial part of the skin. The desquamation marker may be, for example, Kallikrein Related Peptidase 7 (KLK7).

The term “antimicrobial marker” as used herein refers to a marker involved in the defence reaction of the skin against microorganisms. Preferably, the antimicrobial marker is serpin peptidase inhibitor, clade B, member 13 (SERPINB13).

The term “immunity marker” as used herein refers to a marker determining the identity of the organism and defending the latter against the outside. Immunity markers serve as the first line of defence against bacterial infections. Preferably, the immunity marker is selected from the group consisting of calcium binding protein A7 (S100A7) and Toll-like receptor 2 (TLR2).

The term “inflammation marker” as used herein refers to a marker modulated during an inflammatory response in the skin and markers of cells involved in the inflammation process. Preferably, the inflammation marker is selected from the group consisting of interleukins, preferably interleukin 13 receptor, alpha 2 (IL13RA2), or chemokines, preferably Chemokine (C-C motif) ligand 5 (CCL5) of Chemokine (C-C motif) ligand 27 (CCL27).

Preferably, the markers of the disclosed herein can include claudins, including caspase 14 (CASP14), sciellin (SCEL), involucrin (IVL), Small Proline Rich Protein 1B (SPRR1B), Kallikrein Related Peptidase 7 (KLK7), sulfotransferase family, cytosolic, 2B, member 1 (SULT2B1), serpin peptidase inhibitor, clade B, member 13 (SERPINB13), calcium binding protein A7 (S100A7), Toll-like receptor 2 (TLR2), Chemokine (C-C motif) ligand 5 (CCL5), Chemokine (C-C motif) ligand 27 (CCL27) interleukin, including Interleukin 13 receptor, alpha 2 (IL13RA2) etc.

Advantageously, determining whether the expression of a biological marker is modulated involves measuring the expression of the biological marker. The biological marker can be measured by any method known to the skilled person.

The expression of a gene can be measured, for example, at the nucleotide level, by measuring the amount of transcripts of said gene, or at the peptide level, by measuring, for example, the amount of proteins from said transcripts. Thus, “measuring the expression level of a gene” refers herein to the measuring the quantity or cellular concentration of the product of the gene in its peptide form or in its nucleotide form. In particular, the expression of at least one gene (e.g. any of the gene biomarkers disclosed herein) can be measured to determine that the expression of this gene is modulated.

Generally, the expression of the biomarker according to the present disclosure will be detected in vitro from a sample of the subject treated with the bacterial strain, bacterial extract, or composition disclosed herein. Preferably, the sample will be a sample of the subject's skin.

A “skin sample” according to the disclosure comprises any sample from the subject containing skin cells. Advantageously, the skin cells according to the present disclosure comprise normal, healthy or pathological cells, or cells derived from lineages. For example, the cultured skin cells may be cells obtained from skin tissue explant. By “explant” or “skin explant” is meant here a collection of cells or skin tissue, which may be taken for surgical purposes or for analysis. Advantageously, the said skin sample is obtained from the surface of the skin by stripping the said surface.

In order to determine whether the expression of a biological marker is increased or decreased in the skin sample of the subject treated with the bacterial strain, bacterial extract, or composition disclosed herein, the expression level of the marker may be compared with a reference expression level.

Accordingly, determining whether the expression of the biological marker is modulated by the administration of the bacterial strain, bacterial extract, or composition disclosed herein to the subject involves measuring the expression of the marker in a skin sample of the subject and comparing this expression level of the biological marker with a reference expression level.

As used herein, “a reference expression level of a biomarker” refers to any expression level of said marker used as a reference. For example, a reference expression level may be obtained by measuring the expression level of the marker of interest in a skin sample of a healthy subject. Alternatively, the reference expression level of a biomarker corresponds to the expression level of the marker in the sample of the subject's skin in the absence or presence of a particular treatment. For example, in one particular instance, the reference expression level of a biomarker is obtained by measuring the expression of the marker in the skin sample that has not been contacted with an active or formulation. In another particular instance, the expression of the marker is measured in a skin sample from the subject treated with an active or formulation known to be effective against the particular cutaneous inflammatory disorder affecting the subject.

It will also be readily apparent to the skilled person that a meaningful comparison of the expression level of the biological marker with a reference expression level will advantageously involve using a normalisation factor.

This normalisation factor may, for example, be a directly accessible physical marker such as the mass of cells in the sample, or the mass of a cellular constituent, such as the mass of cellular DNA or the mass of cellular proteins.

It may also be advantageous to use as a normalisation factor the expression level of a gene that is expressed at the same level in all, or nearly all, cells of the organism. In other words, according to a particular instance, the expression level of a housekeeping gene is used as the normalisation factor. According to another instance, the expression level of the biological marker and the reference expression level are normalised using the expression level, not of the housekeeping genes, but of the proteins encoded by them. A housekeeping gene is a gene expressed in all cell types, which encodes a protein with a basic function necessary for the survival of all cell types. A list of human housekeeping genes can be found in Eisenberg et al (Trends in Genet, 19:362-365, 2003). Examples of housekeeping genes include, notably, RPS28, GAPDH, B2M, TFRC, YWHAZ, RPLO, 18S, GUSB, UBC, TBP, GAPDH, PPIA, POLR2A, ACTB, PGK1, HPRT1, IPO8 and HMBS.

The disclosure will be better understood by reading the examples below which illustrate it without limiting its scope.

### EXAMPLE 1: CHARACTERISATION OF THE BACTERIAL STRAIN LMB275

In order to characterise the bacterial strain LMB275, its genomic composition and growth conditions were determined and compared to the three closest known bacterial strains: Aquabacterium parvum B6T (DSM 11968T), Aquabacterium commune B8T (DSM 11901T), and Aquabacterium citratiphilum B4T (DSM 11900T).

Extraction and Sequencing of 16 rRNA of the Bacterial Strain LMB275

Genomic DNA was extracted with the Maxwell® 16 MDx Instrument (AS3000, Promega) from a 2-ml liquid culture with the Maxwell® 16 LEV Blood DNA kit (Promega, Charbonnières-les-Bains, France) including an initial lysis step as described by Fagervold et al. (2020). PCR (polymerase chain reaction) targeting the 16S rRNA gene was performed with universal bacterial primers 27Fmod (SEQ ID NO 2: 5′ AGRGTTTGATCMTGGCTCAG-3′) (Eiler & Bertilsson, 2004) and 1492Rmod (SEQ ID NO 3: 5′-TACGGYTACCTTGTTAYGACTT-3′) (Acinas et al. 2005). The thermal cycling program started at 94° C. for 5 min, followed by 30 cycles of 94° C. for 15 s, 50° C. for 15 s and 72° C. for 15 s, and by a final step of 72° C. for 1.5 min. The PCR product was purified using a PCR clean up kit ExoSap IT (Applied Biosystems), and then sequenced using primers 907R (SEQ ID NO 4: 5′-CCGTCAATTCCTTTGAGTTT-3′) and S8 (SEQ ID NO 5: 5′-GTAGCGGTGAAATGCGTAGA-3′), the BigDye Terminator Cycle Sequencing kit (Applied Biosystems) with an ABI Prism 3130xl automated DNA analyser (Applied Biosystems).

A nearly complete sequence (1460 bp) of the 16S rRNA (ribosomal ribonucleic acid) gene of strain LMB275 was obtained and was registered as Genbank accession N° ON107272. This sequence corresponds to the sequence identified as SEQ ID NO: 1. This gene sequence was compared to those in EzBioCloud (Yoon et al. 2017). Multiple sequence alignments were performed using the BioEdit software version 7.2.5 (Hall, 1999). Phylogenetic trees were reconstructed by the neighbour-joining, maximum-likelihood and maximum-parsimony methods using mega X software (Kumar et al. 2018). Evolutionary distances for the NJ (neighbour joining), ML (multilabel learning) and MP (multi-packing) trees were computed using the Tamura three-parameters model (Tamura, 1992). In each case, bootstrap values were calculated based on 1000 replications.

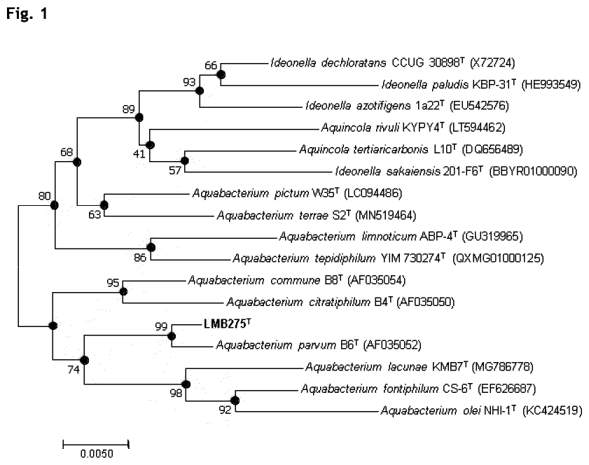

Initial comparison of 16S rRNA gene sequence within the EzBioCloud online database assigned strain LMB275 into the genus Aquabacterium and showed the highest sequence similarity with Aquabacterium parvum DSM 11968T (99.1%). Further, close phylogenetic neighbours of strain LMB275 were Aquabacterium commune DSM 11901T and Aquabacterium citratiphilum DSM 11900T with 97.9 and 97.0% similarities respectively. The relationship of strain LMB275 and all members of the genus Aquabacterium was also supported by the phylogenetic analysis based on 16S rRNA sequence and including gene sequences of all validly published Aquabacterium species. This analysis confirmedA. parvum as the closest phylogenetic neighbour of LMB275 and showed that they formed together a unique lineage strongly supported by bootstrap value and that distinguished them from other members Aquabacterium genus (FIG. 1). The same result was obtained when the tree was reconstructed using the neighbour-joining, the minimum-evolution and the maximum likelihood algorithms.

Phylogenetic Tree Based of 16S rDNA Gene Sequences Showing the Relationships of Strain LMB275 with the Related Aquabacterium Species.

DNA-DNA hybridisation was carried out by the DSMZ Identification Service (Leibniz-Institut DSMZ-Deutsche Sammlung von Mikroorganismen und Zellkulturen GmbH, Braunschweig, Germany) to determine the level of relatedness between the strain LMB275 and the closest relativeA. parvum DSM 11968T. After DNA purification by chromatography on hydroxyapatite according to Cashion et al. (1977), DNA-DNA hybridisation was carried out in duplicate according to the thermal denaturation and renaturation method (De Ley et al. 1970; Huss et al, 1983) using a Cary spectrophotometer equipped with a Peltier temperature controller with an in-situ temperature probe (Varian). The DNA-DNA hybridisation value was 47.3% DNA-DNA similarity (39.7% for duplicate), far below the threshold value of 70% recommended for the definition of bacterial species and thus clearly indicating that strain LMB275 does not belong to the closest phylogenetic relative speciesA. parvum species.

### Growth Conditions

The temperature range for growth was tested on a series of modified R2A plates incubated to temperature starting to 4° C. up to 45° C. NaCl tolerance was investigated using modified R2A broth with various NaCl concentrations (0 to 5%, w/v). The pH range for growth (pH 5.0 to 10.0 at intervals of 1.0 pH units) was determined in modified R2A broth that was buffered with MES (2-(N-morpholino) ethanesulfonic acid), HEPES (4-(2-hydroxyethyl)-1-piperazineethanesulfonic acid), or AMPSO (3-([1,1-Dimethyl-2-hydroxyethyl]amino)-2-hydroxypropanesulfonic acid) buffer (Sigma). The concentration of bacterial cells in liquid cultures was assessed with an OD Scanner (System-c bioprocess) seven days. The strain grew optimally at 30-37° C., pH 7.0 and with no NaCl but was able to grow at 10 to 45° C. (but not 4° C.), at 6.0 to 10.0 pH units (but not 5.0), and was not able to grow in all other NaCl concentrations tested.

The capacity of the strain to grow at 30° C. on nutrient-rich media was tested on Trypticase Soy Agar (TSA, Sigma) and on nutrient agar containing 3 g beef extract, 5 g peptone, 5 g NaCl, and 15 g agar per litre. Colonies of the strain LMB275 formed onto both media.

Growth under aerobic, anaerobic, microaerophilic, and under 5% CO₂ conditions was determined on modified R2A agar using GENbag anaer, GENbag microaer, and GENbag C₀2 systems, respectively (BioMérieux) after incubating the strain for 2 days at 30° C. Optimal growth was obtained aerobically and under microaerophilic condition. Weak growth was observed with 5% C₀2, and no growth occurred under anaerobic conditions.

The ability of strain LMB275 to reduce different inorganic electron acceptors in anaerobic condition (anaerobic chamber) was assessed in mineral deep agar medium (0.66 g (NH₄)₂SO₄; 1.36 g KH₂PO₄; 0.123 g MgSO₄.7H₂0; 0.031 g CaCl₂) and 0.018 g FeCl₂ per litre) supplemented with 5 mM sodium acetate and one of the following compounds: 0.1% (w/v) KNO₃, 0.2% (w/v) KNO₂, 10 mM Na₂SO₄, 10 mM NaClO₃ or 10 mM Fe (III)-citrate as electron acceptors (Kalmbach et al., 1999). Briefly, 0.2 ml of fresh liquid culture were inoculated in 10 ml in liquefied and tempered medium. LMB275 grew using nitrate, chlorate, sulfate and iron (III), but no reduction of nitrite was observed.

Polar lipids were extracted from freeze-dried cell material using a choroform:methanol: 0.3% aqueous NaCl mixture (modified after Bligh and Dyer, 1959) and were analysed as described by Tindall (2007). The polar lipid pattern consisted of a mixture of phosphatidylethanolamine as the major component, phosphatidylglycerol, diphosphatidyl-glycerol and several uncharacterised phospholipids.

The sensitivity of the bacterial strain to antibiotics was tested by the disc diffusion method after spreading cell suspensions (8.0 McFarland) on modified R2A agar plates. The antibiotics and their concentration used on filter-paper discs for the susceptibility assay are listed in the species definition. The effect of antibiotics on cell growth was assessed after 48 and 72 h at 30° C. The bacterial strain is resistant to cefotaxim (30 μg), polymixin B (300 UI), ampicillin (10 μg), penicillin (10 UI) and amoxicillin (30 μg) and sensitive to chloramphenicol (30 μg), ciprofloxacin (5 μg), vancomycin (30 μg), rifampicin (30 μg), imipenem (10 μg), ofloxacin (5 μg), kanamycin (30 μg), tetracyclin (30 μg), erythromycin (15 μg), streptomycin (10 μg).

In summary, strain LMB275 can be distinguished from the related species of the genus Aquabacterium by its physiological and biochemical characteristics as well as by its phylogenetic position and DNA-DNA relatedness.

### EXAMPLE 2: CULTURE OF BACTERIAL STRAIN LMB275

An example of a culture process is described below. This example is only illustrative and should in no way be considered limitative of the various ways of growing LMB275.

### 2.1. Obtention of the Growth Medium

The composition of the medium is shown below in Table 1.

The growth medium is prepared by dissolving the various constituents in distilled water. The pH is then adjusted to 7 before sterilisation at 121° C. for 20 minutes.

TABLE 1
Composition of the growth medium

| Chemicals | Unit | Concentration |
|---|---|---|
| NH₄Cl | g/L | 1 |
| K₂HPO₄ | g/L | 0.54 |
| KH₂PO₄ | g/L | 0.26 |
| CaCl₂, 2H₂O | g/L | 0.0147 |
| MgSO₄, 7H₂O | g/L | 0.247 |
| Tween 80 | ml/L | 1 |
| Urea | g/L | 0.6 |
| Sodium Acetate | g/L | 4.5 |
| Yeast extract | g/L | 2 |

### 2.2. Obtention of the Pre-Culture

A pre-culture is performed by adding 1.5 mL of cryopreserved cell suspension to 100 ml of medium culture whose composition is described above. The suspension is incubated at 25° C. under agitation at 110 rpm for 2.5 days.

### 2.3. Obtention of the Bacterial Culture

Ten mL of the preculture are then inoculated in 250 ml of medium and incubated at 25° C. under agitation at 100 rpm. Nine such cultures are thus set up. When the beginning of stationary phase is reached (i.e., after ca. 38 hours), the nine cultures are mixed for homogenisation, thereby constituting the bacterial culture which is used for extraction.

### EXAMPLE 3: EXTRACTION OF THE FRACTIONS L0, BL0, S0 AND ES0

The different fractions or extracts L0, BL0, S0 and ES0 are then prepared from the bacterial culture obtained in example 2.

### 3.1. Extraction L0

Three successive extractions with ethyl acetate were performed on 1.8 L of bacterial culture comprising the bacterial cells, using a total of 1.8 L of ethyl acetate. At the end of the extraction, the aqueous phase was discarded, and the organic phase evaporated using a rotary evaporator. The extract was then aliquoted, dried, and stored at −80° C. A total of 256.6 mg of lipophilic extract is obtained.

The extract L0 thus obtained contains ethyl acetate-soluble lipophilic biomolecules.

### 3.2. Extraction BL0

Three successive extractions were performed with butanol on 2 L of bacterial culture comprising the cell suspension, using a total of 2L of butanol. At the end of the extraction, the aqueous phase was discarded, and the organic phase evaporated using a rotary evaporator. The extract was then aliquoted, dried, and stored at −80° C. A total of 533 mg of lipophilic extract was obtained.

The extract BL0 contains lipophilic biomolecules soluble in butanol.

### 3.3. Extraction S0

Fifty mL of the bacterial culture were centrifuged at 10000 g for 20 minutes at 4° C. The supernatant was recovered. After 25 ml of 900 mM L-Arginine buffer were added, the supernatant was filtered on syringe filters with 0.22 μm Polyethersulfone membrane and then aliquoted and stored at −20° C. The protein concentration in the S0 extract, determined with the BCA kit (Pierce), was 369 μg/ml, for a total of about 28 mg of protein for 50 ml of bacterial culture used. The extract S0 contains components from the growth medium, biomolecules excreted by the strain in the supernatant and L-Arginine at 300 mM.

### 3.4. Extraction ES0

A volume of 50 ml of 900 mM L-Arginine buffer was added to 100 ml of bacterial culture and the mixture is stirred at 500 rpm with a magnetic bar at 4° C. for 1 hour. The suspension was then centrifuged at 10000 g for 20 min at 4° C. The supernatant was filtered on syringe filters with a 0.22 μm Polyethersulfone membrane, aliquoted and stored at −20° C. The protein concentration in the ES0 extract, determined with the BCA kit (Pierce), was 380 μg/ml, for a total of about 57 mg of protein for 100 mL of bacterial culture used.

The extract ES0 contains components from the growth medium, bacterial biomolecules secreted in the medium, periplasmic biomolecules extracted by L-Arginine buffer at 300 mM and L-Arginine at 300 mM.

### 3.5 Chemical Profile of the Extracts

A liquid chromatography coupled to tandem mass spectrometry was made with the extracts L0 and BL0.

### LC/MS-MS Protocol

To analyse the extracts, MS-MS analysis were performed with a Q Exactive Focus Orbitrap System and a Corona VEO RS detection coupled to an Ultimate 3000TM UHPLC system (Thermo Fisher Scientific). Chromatography was performed on a Luna Omega polar C18 column (150x2.1 mm, 1.6 μm; Phenomenex), at 42° C. using a 0.5 mL/min flow rate. Water (eluent A) and acetonitrile (eluent B), each containing 0.1% formic acid, were used for gradient elution. Gradient elution was achieved starting at 20% of B for 3 minutes before injection and 1 minute after, then from 1 to 7 minutes increasing B to 100% (curve 2) and stay at that rate for 8 more minutes. For the detection of the metabolites, we performed the analysis in electrospray positive ionization mode in the 133-2000 m/z range in discovery mode. 4 μL of extracts are injected at 1.5 mg/mL.

### SDS-PAGE Protocol

5 μl of ES0 and S0 extracts were denatured for 5 min at 94° C. with an equal volume of 2X Laemmli buffer (Bio-Rad) and then resolved on a 4-15% Mini-PROTEAN® TGX™ Precast Protein Gel (Bio-Rad) in Tris-glycine-SDS buffer (Bio-Rad) under 150 V for 45 min. The gel was then silver stained to visualise proteins patterns.

The result shows that these two extracts are composed by different types of molecules.

A sodium dodecyl sulfate polyacrylamide gel electrophoresis was made with the extracts S0 and ES0. The result shows that these two extracts are composed by different types of proteins.

### EXAMPLE 4: PREPARATION OF HUMAN EPIDERMAL KERATINOCYTES (NHEK)

The effects of the bacterial extracts prepared in example 3 (i.e., L0, BL0, S0 and ES0) on gene expression were assessed with normal human epidermal keratinocytes (NHEK) which have been stimulated by an inflammatory cytokine mix (IL-4+IL-13+IL-22+TNF-α), a particularly useful model representative of the chronic phase of atopic dermatitis.

### 4.1. Culture and Treatment

### Pre-Incubation Step

Keratinocytes were seeded in 24-well plates and grown for 24 hours in growth medium with culture conditions at 37° C. and 5% C₀2. The growth medium comprised Keratinocyte SFM (Serum Free Medium) supplemented with Epidermal Growth Factor (EGF) 0.25 ng/ml, Pituitary extract (PE) 25 μg/ml and Gentamycin 25 μg/ml. The medium was then removed and replaced by assay medium containing either no added extract or compound (negative control), one of the bacterial extracts of example 3 (L0, BL0, S0 or ES0), or the reference compound (JAK Inhibitor | at 10 μM). Cells were then pre-incubated for 24 hours. The assay medium comprised Keratinocyte SFM supplemented with Gentamycin 25 μg/ml.

### Cytokine Stimulation Step

After pre-incubation, the assay medium was renewed, the mix of cytokines (IL-4+IL-13+TNF-α +IL-22, each at 10 ng/ml) added and the cells then incubated for 24 hours. Cytokine stimulation induces a phenotype in NHEK which is similar to the one observed during the chronic phase of atopic dermatitis (Bernard et al., 2012). In parallel, non-stimulated controls are performed.

All experiments were performed in triplicate (n=3).

At the end of the incubation time, the culture supernatants were collected and stored at −80° C. for potential further analysis. The cells were washed in phosphate buffered saline (PBS) solution and immediately frozen at 80° C.

### RNA Extraction

Cells from the replicates of the same experimental condition were pooled.

Total RNA was extracted with TriPure Isolation Reagent® (Roche Molecular System Inc.) according to the supplier's instruction. The complementary DNA (cDNA) was synthetised by reverse transcription of total RNA in presence of oligo (dT) and Transcriptor Reverse Transcriptase (Roche Molecular System Inc.).

### 4.2. Differential Expression Analysis Step

RT-qPCR (real time quantitative PCR) was used to analyse the expression of markers in total RNA extracted from the cell monolayers of each experimental condition. The LightCycler® system (Roche Molecular System Inc.) was used according to the supplier's instruction,

Two reference genes (GAPDH and RPS28) were used in the PCR array for data normalisation because of their constitutive expression. Consequently, the level of expression of the target markers was compared to the mean expression level of these 2 markers for all test conditions.

Different genes markers are reported:
barrier function, differentiation, lipid and desquamation markers: coding gene of Small Proline Rich Protein 1B (SPRR1B), coding gene of Kallikrein Related Peptidase 7 (KLK7), coding gene of Involucrin (IVL), coding gene of Caspase 14 (CASP14), coding gene of Sciellin (SCEL) and coding gene of sulfotransferase family, cytosolic, 2B, member 1 (SULT2B1);
antimicrobial and innate immunity markers: coding gene of serpin peptidase inhibitor, clade B, member 13 (SERPINB13), coding gene of calcium binding protein A7 (S100A7), coding gene of Toll-like receptor 2 (TLR2);
inflammation markers: coding gene of Chemokine (C-C motif) ligand 5 (CCL5), coding gene of Chemokine (C-C motif) ligand 27 (CCL27) and coding gene of interleukin 13 receptor, alpha 2 (IL13RA2).

### EXAMPLE 5: EFFECTS OF THE DIFFERENT EXTRACTS ON THE GENE EXPRESSION PATTERN IN KERATINOCYTES UNDER IL-4+IL-13+TNF-α +IL-22 CYTOKINE MIX STIMULATION

### 5.1. Validation of IL-4+IL-13+IL-22+TNF-α Stimulation on the Gene Expression Pattern in Keratinocytes

Cytokine stimulation is known to induce a phenotype in NHEK which is representative of the chronic phase of atopic dermatitis (see e.g., Bao et al., 2013; Smith et al., 2021). The use of JAK inhibitor counteracts the effect of cytokines mix by representing an inhibition of the inflammation.

Table 2 shows that the treatment of NHEK with the cytokine mix (IL-4+IL-13+TNF-α +IL-22, each at 10 ng/ml) for 24 hours induced a strong increase in the expression of inflammatory markers and antimicrobial peptides. importantly, the latter markers are considered biomarkers of atopic dermatitis. The effect of the cytokine mix on the differentiation markers is less pronounced with an inhibition of some markers and an up-regulation for others resulted in an increased expression of innate immunity markers (mainly S100A7, and TLR2), chemokine markers (CCL5) and a strong increase in the expression of the cytokine receptor marker IL13RA2. This pro-inflammatory effect of the cytokine mix was associated to a dedifferentiating effect as observed by the decreased expression of keratinocyte differentiation markers (inhibition of SPRR1B, IVL, CASP14) and the up regulation of marker SERPINB13 involved in proliferation.

When the reference compound, JAK Inhibitor I, was added at 10 UM, it completely inhibited the stimulating effects of the cytokine mix by down-regulating the expression of inflammatory markers and innate immunity markers (TLR2). on the other hand, JAK Inhibitor I led to an increase in the expression of keratinocyte differentiation markers (including SPRR1B and mainly IVL).

This typical gene expression pattern induced by the cytokine mix stimulation and the effect of the reference compound were as expected, thereby validating the assay (Bernard et al., 2012).

### 5.2. Effects of L0 on Gene Expression in Cytokine-Stimulated Keratinocytes

As shown in Table 2, L0 added at 35 μg/ml induced in stimulated keratinocytes a marked increase of most of the markers involved in keratinocyte differentiation. Even though antimicrobial markers were not as strongly induced as with the cytokine mix alone, their expression was nonetheless clearly increased in the presence of L0, notably for SERPINB13, S100A7, and TLR2, showing that L0 have a strong effect on antimicrobial markers. These modulations were associated with a strong decrease of the inflammation markers such as CCL27 and IL13RA2 corresponding to a low expression level.

TABLE 2
Comparison of gene expression pattern
in keratinocytes of extract L0

| | | Cytokines | Cytokine | Cytokine |
|---|---|---|---|---|
| | | mix (IL-4 + | mix 10 | mix 10 |
| | | IL-13 + | ng/ml + | ng/ml + |
| | | TNF-α + | JAK | Extract L0 |
| | | IL-22) | inhibitor I | 35 μg/ml |
| | | 10 ng/ml | 10 μM | L0 |
| | Genes | (% control | (% control | (% control |
| | abbreviation | Mean HK) | Mean HK) | Mean HK) |
| Housekeeping | GAPDH | 98 | 99 | 105 |
| | RPS28 | 107 | 102 | 83 |
| Differentiation/ | SPRR1B | 41 | 557 | 1687 |
| Barrier | KLK7 | 89 | 146 | 416 |
| function/Lipid/ | IVL | 25 | 986 | 836 |
| Desquamation | CASP14 | 33 | 199 | 618 |
| | SCEL | 86 | 141 | 371 |
| | SULT2B1 | 181 | 231 | 2519 |
| Antimicrobial | SERPINB13 | 354 | 27 | 204 |
| peptides, | S100A7 | 1925 | 94 | 1006 |
| innate immunity | TLR2 | 1180 | 21 | 135 |
| Inflammation | CCL5 | 662 | 23 | 74 |
| | CCL27 | 2136 | 72 | 66 |
| | IL13RA2 | 2339 | 12 | 212 |

### 5.3. Effects of BL0 on Gene Expression in Cytokine-Stimulated Keratinocytes

The effects of BL0 at 35 μg/ml on gene expression in stimulated keratinocytes are shown in Table 3. A slight modulation of the expression of genes involved in keratinocyte differentiation was induced by BL0: KLK7, and SULT2B1 were up-regulated. On the other hand, BL0 induced a strong decrease of the inflammation markers CCL27 and IL13RA2. Even though antimicrobial markers were not as strongly induced as with the cytokine mix alone, their expression was nonetheless clearly increased in the presence of BL0, notably for SERPINB13, S100A7, and TLR2, showing that BL0 have a strong effect on antimicrobial markers.

TABLE 3
Comparison of gene expression pattern
in keratinocytes of extract BL0

| | | Cytokine | Cytokine | |
|---|---|---|---|---|
| | | mix (IL-4 + | mix 10 | Cytokine |
| | | IL-13 + | ng/ml + | mix 10 |
| | | TNF-α + | JAK | ng/ml + |
| | | IL-22) | inhibitor I | Extract BL0 |
| | | 10 ng/ml | 10 μM | 35 μg/ml |
| | Genes | (% control | (% control | (% control |
| | abbreviation | Mean HK) | Mean HK) | Mean HK) |
| Housekeeping | GAPDH | 98 | 99 | 101 |
| | RPS28 | 107 | 102 | 96 |
| Differentiation/ | SPRR1B | 41 | 557 | 113 |
| Barrier | KLK7 | 89 | 146 | 231 |
| function/Lipid/ | IVL | 25 | 986 | 126 |
| Desquamation | CASP14 | 33 | 199 | 138 |
| | SCEL | 86 | 141 | 8 |
| | SULT2B1 | 181 | 231 | 340 |
| Antimicrobial | SERPINB13 | 354 | 27 | 67 |
| peptides, | S100A7 | 1925 | 94 | 342 |
| innate immunity | TLR2 | 1180 | 21 | 114 |
| Inflammation | CCL5 | 662 | 23 | 139 |
| | CCL27 | 2136 | 72 | 30 |
| | IL13RA2 | 2339 | 12 | 66 |

### 5.4. Effects of S0 on Gene Expression in Cytokine-Stimulated Keratinocytes

Table 4 summarises the effects of gene expression of S0 added at 10 μg/ml to stimulated keratinocytes. Most of the markers involved in keratinocyte differentiation, and tight junctions, were induced by S0. Even though antimicrobial markers were not as strongly induced as with the cytokine mix alone, their expression was nonetheless clearly increased in the presence of S0, notably for SERPINB13, S100A7, and TLR2, showing that S0 have a strong effect on antimicrobial markers. These modulations were associated with a strong decrease of the inflammation markers CCL5 and CCL27.

TABLE 4
Comparison of gene expression pattern
in keratinocytes of extract S0

| | | Cytokine | Cytokine | |
|---|---|---|---|---|
| | | mix (IL-4 + | mix 10 | Cytokine |
| | | IL-13 + | ng/ml + | mix 10 |
| | | TNF-α + | JAK | ng/ml + |
| | | IL-22) | inhibitor I | Extract S0 |
| | | 10 ng/ml | 10 μM | 10 μg/ml |
| | Genes | (% control | (% control | (% control |
| | abbreviation | Mean HK) | Mean HK) | Mean HK) |
| Housekeeping | GAPDH | 98 | 99 | 106 |
| | RPS28 | 107 | 102 | 78 |
| Differentiation/ | SPRR1B | 41 | 557 | 618 |
| Barrier | KLK7 | 89 | 146 | 170 |
| function/Lipid/ | IVL | 25 | 986 | 922 |
| Desquamation | CASP14 | 33 | 199 | 225 |
| | SCEL | 86 | 141 | 330 |
| | SULT2B1 | 181 | 231 | 672 |
| Antimicrobial | SERPINB13 | 354 | 27 | 88 |
| peptides, | S100A7 | 1925 | 94 | 867 |
| innate immunity | TLR2 | 1180 | 21 | 76 |
| Inflammation | CCL5 | 662 | 23 | 25 |
| | CCL27 | 2136 | 72 | 43 |
| | IL13RA2 | 2339 | 12 | 125 |

### 5.5. Effects of ES0 on Gene Expression in Cytokine-Stimulated Keratinocytes

As shown in Table 5, ES0 (10 μg/ml), induced a strong increase in stimulated keratinocytes of most of the markers involved in keratinocyte differentiation (protein and lipid markers). Even though antimicrobial markers were not as strongly induced as with the cytokine mix alone, their expression was nonetheless clearly increased in the presence of ES0, notably for SERPINB13, S100A7, and TLR2, showing that ES0 have a strong effect on antimicrobial markers. These modulations were associated with a slight decrease of the inflammation markers CCL5 and CCL27.

TABLE 5
Comparison of gene expression pattern
in keratinocytes of extract ES0

| | | Cytokine | Cytokine | |
|---|---|---|---|---|
| | | mix (IL-4 + | mix 10 | Cytokine |
| | | IL-13 + | ng/ml + | mix 10 |
| | | TNF-α + | JAK | ng/ml + |
| | | IL-22) | inhibitor I | Extract ES0 |
| | | 10 ng/ml | 10 μM | 10 μg/ml |
| | Genes | (% control | (% control | (% control |
| | abbreviation | Mean HK) | Mean HK) | Mean HK) |
| Housekeeping | GAPDH | 98 | 99 | 98 |
| | RPS28 | 107 | 102 | 106 |
| | SPRR1B | 41 | 557 | 643 |
| Differentiation/ | KLK7 | 89 | 146 | 200 |
| Barrier | IVL | 25 | 986 | 749 |
| function/Lipid/ | CASP14 | 33 | 199 | 112 |
| Desquamation | SCEL | 86 | 141 | 264 |
| | SULT2B1 | 181 | 231 | 531 |
| Antimicrobial | SERPINB13 | 354 | 27 | 113 |
| peptides, | S100A7 | 1925 | 94 | 985 |
| innate immunity | TLR2 | 1180 | 21 | 60 |
| Inflammation | CCL5 | 662 | 23 | 24 |
| | CCL27 | 2136 | 72 | 53 |
| | IL13RA2 | 2339 | 12 | 153 |

As mentioned above, cytokine stimulation of NHEK leads to a phenotype which is representative of the chronic phase of atopic dermatitis. This effect is suppressed by the addition e of JAK inhibitor I, which mimics the inhibition of the inflammation.

The inventors have observed that JAK inhibitor I induces an increase of keratinocyte differentiation markers (protein and lipid markers) and a decrease of inflammation markers and of antimicrobial and innate immunity markers.

All four bacterial extracts tested (L0, BL0, S0 and ES0) present the same pattern of effects on gene expression as JAK inhibitor I: expression of keratinocyte differentiation markers (protein and lipid markers) is increased, whilst the expression of inflammation markers is reduced. Surprisingly, addition of any one of these bacterial extracts leads to a decrease of inflammation of in the cytokine-stimulated keratinocyte model of atopic dermatitis.

In fact, during their differentiation, keratinocytes produce various constituents by allowing the thickening of the epidermis to ensure the integrity of the barrier function of the skin. Overexpression of differentiation marker genes results in the stimulation and increase in keratinocyte differentiation and consequently maintaining the skin barrier. The skin barrier protects against transcutaneous water loss and the penetration of microorganisms or environmental antigens. Lipids and corneocytes in the stratum corneum form an impermeable barrier and prevent the loss of water through the skin.

Keratinocytes assume their role as an antimicrobial barrier, notably by expressing antimicrobial peptides in the atopic dermatitis. The antimicrobial peptides, innate immunity are biomarkers of atopic dermatitis and psoriasis. An overexpression of this markers corresponds to the expression in the case of atopic dermatitis. As described, keratinocytes treated with one of the four extracts expressed antimicrobial markers demonstrate a reducing of antimicrobial biomarkers expressions assimilated a protective and anti-inflammatory effect.

Therefore, the inventors have demonstrated that the use of bacterial extracts induce an inhibition of the cutaneous inflammation in particular in cutaneous inflammatory disorders by stimulating and maintaining the skin barrier formed by keratinocytes. These bacterial extracts allow the stimulation of keratinocytes and their proliferation enabling to prevent water loss and to maintain thickening of the epidermis to its optimal functionality i.e., skin barrier function.

### BIBLIOGRAPHIC REFERENCES

Bieber T. 2010. Atopic dermatitis. Ann Dermatol. May;22 (2): 125-37.
Michael S Waterman, Temple F Smith 1981, Comparison of biosequences, Advances in Applied Mathematics, Volume 2, Issue 4, Pages 482-489.
Needleman S B, Wunsch C D. 1970, A general method applicable to the search for similarities in the amino acid sequence of two proteins. J Mol Biol. Mar;48 (3): 443-53.
Pearson W R, Lipman D J. 1988, Improved tools for biological sequence comparison. Proc Natl Acad Sci USA. Apr;85 (8): 2444-8.
Tatusova et al. 1999, Blast 2 sequences-a new tool for comparing protein and nucleotide sequences, FEMS Microbiol Lett. May 15, 174:247-250.
Tokura Y, Hayano S. 2022, Subtypes of atopic dermatitis: From phenotype to endotype. Allergol Int. Jan;71 (1): 14-24.
Dagnelie M A, Corvec S, Saint-Jean M, Nguyen J M, Khammari A, Dréno B. 2019, Cutibacterium acnes phylotypes diversity loss: a trigger for skin inflammatory process. J Eur Acad Dermatol Venereol. Dec;33 (12): 2340-2348.
Raharja A, Mahil S K, Barker J N. 2021, Psoriasis: a brief overview. Clin Med (Lond). May;21 (3): 170-173.
Van Zuuren E J, Arents BWM, van der Linden MMD, Vermeulen S, Fedorowicz Z, Tan. J 2021. Rosacea: New Concepts in Classification and Treatment. Am J Clin Dermatol. Jul;22 (4): 457-465.
Katta R. 2000. Lichen planus. Am Fam Physician. June 1;61 (11): 3319-24, 3327-8. Erratum in: Am Fam Physician 2000 Oct. 15;62 (8): 1786.
Gupta S, Jawanda M K. 2015. Oral Lichen Planus: An Update on Etiology, Pathogenesis, Clinical Presentation, Diagnosis and Management. Indian J Dermatol. May-Jun;60 (3): 222-9.
Clark G W, Pope S M, Jaboori K A. 2015. Diagnosis and treatment of seborrheic dermatitis. Am Fam Physician. February 1;91 (3): 185-90.
Fagervold S K, Rohée C, Rodrigues AMS, Stien D, Lebaron P 2020. Efficient degradation of the organic U V filter benzophenone-3 by Sphingomonas wittichii strain BP14P isolated from WWTP sludge. STOTEN 758.
Eiler A, Bertilsson S 2004. Composition of freshwater bacterial communities associated with cyanobacterial blooms in four Swedish lakes. Environ Microbiol 6, 1228-1243.
Acinas S G, Sarma-Rupavtarm R, Klepac-Ceraj V, Polz M F 2005. PCR-induced sequence artifacts and bias: insights from comparison of two 16S rRNA clone libraries constructed from the same sample. Appl Environ Microbiol 71, 8966-8969.
Yoon S H, Ha S M, Kwon S, Lim J, Kim Y, Seo H, Chun J 2017. Introducing EzBioCloud: A taxonomically united database of 16S rRNA and whole genome assemblies. Int J Syst Evol Microbiol 67:1613-1617.
Hall T A 1999. BioEdit: a user-friendly biological sequence alignment editor and analysis program for Windows 95/98/N T. Nucl Acids Symp Ser 41:95-98.
Kumar S, Stecher G, Li M, Knyaz C, Tamura K 2018. MEGA X: Molecular evolutionary genetics analysis across computing platforms. Mol Biol Evol 35:1547-1549.
Cashion P, Hodler-Franklin M A, McCully J, Franklin M 1977. A rapid method for base ratio determination of bacterial DNA. Anal Biochem 81:461-466.
De Ley J, Cattoir H, Reynaerts A 1970. The quantitative measurement of DNA hybridisation from renaturation rates. Eur J Biochem 12:133-142
Kalmbach S, Manz W, Wecke J, Szewzyk U. Aquabacterium gen. nov., with description of Aquabacterium citratiphilum sp. nov., Aquabacterium parvum sp. nov, and Aquabacterium commune sp. nov., three in situ dominant bacterial species from the Berlin drinking water system 1999. Int J Syst Bacteriol 2:769-77.
Bligh E G, Dyer W J 1959. A rapid method of total lipid extraction and purification. Can J Biochem Physiol, 37, 911-917.
Tindall B J, Sikorski J, Smibert R M, Kreig N R 2007. Phenotypic characterisation and the principles of comparative systematics. In Methods for General and Molecular Microbiology 3rd edn. Pp. 330-393.
Bernard F X, Morel F, Camus M, Pedretti N, Barrault C, Garnier J, Lecron J C. 2012. Keratinocytes under Fire of Proinflammatory Cytokines: Bona Fide Innate Immune Cells Involved in the Physiopathology of Chronic Atopic Dermatitis and Psoriasis. J Allergy (Cairo).; 2012:718725.

Bao L, Zhang H, Chan L S. 2013. The involvement of the JAK-STAT signaling pathway in chronic inflammatory skin disease atopic dermatitis. JAKSTAT. July 1;2 (3): e24137.
Smith P, Yao W, Shepard S, Covington M, Lee J, Lofland J, Naim A, Sheth T, Parikh B, Yeleswaram S. 2021. Developing a JAK Inhibitor for Targeted Local Delivery: Ruxolitinib Cream. Pharmaceutics. July 8;13 (7).

1. Bacterial strain, wherein the bacterial strain has been deposited with the CNCM (Institut Pasteur, Paris, France) on Nov. 4, 2022, under the reference I-5858.

2. A method for preparing an extract of the bacterial strain according to claim 1, wherein the method comprises:
a) a bacterial culturing step comprising growing the bacterial strain in a suitable growth medium thereby obtaining a bacterial culture,
b) an extraction step comprising adding an organic solvent or a basic buffer to the bacterial culture of step a),
thereby obtaining an extract of the bacterial strain.

3. The method according to claim 2, wherein the extraction step b) comprises the further steps of:
b1) separating the liquid and solid phases,
b2) adding the basic buffer to the liquid phase, and
b3) recovering the buffered liquid phase.

4. The method according to claim 2, wherein the extraction step b) comprises the further steps of:
b1′) adding the basic buffer to the bacterial culture,
b2′) separating the liquid and solid phases, and
b3′) recovering the buffered liquid phase.

5. The method according to claim 3, further comprising a step c) of filtrating the buffered liquid phase of step b3) or b3′).

6. The method according to claim 2, wherein the basic buffer is an arginine buffer.

7. The method according to claim 2, wherein the extraction step b) comprises the further steps of:
b1″) adding the organic solvent to the bacterial culture,
b2″) separating the aqueous and organic phases, and
b3″) recovering the organic phase.

8. The method according to claim 2, wherein the organic solvent of step b) is alcohol (C1-C6) or alkyl acetate.

9. The method according to claim 8, wherein the alcohol is butanol.

10. The method according to claim 8, wherein the alkyl acetate is ethyl acetate.

11. A bacterial extract obtainable according to the method of claim 2.

12. A composition comprising the bacterial strain according to claim 1, or an extract thereof.

13. The composition of claim 12, wherein the bacterial extract is obtainable according to a method which comprises:
a) a bacterial culturing step comprising growing the bacterial strain in a suitable growth medium thereby obtaining a bacterial culture,
b) an extraction step comprising adding an organic solvent or a basic buffer to the bacterial culture of step a), thereby obtaining an extract of the bacterial strain.

14. Composition according to claim 12, wherein the composition is suitable for topical application.

15. A method for preventing or treating cutaneous inflammatory disorders, comprising administering to a subject in need thereof an effective amount of the bacterial strain according to claim 1.

16. The method according to claim 15, wherein the cutaneous inflammatory disorder is selected among atopic dermatitis pruritis, psoriasis, eczema, rosacea, lichen planus, seborrheic dermatitis or acne.

17. The method according to claim 2, wherein the extraction step b) comprises the further steps of:
b1″) adding the organic solvent to the bacterial culture,
b2″) separating the aqueous and organic phases, and
b3″) recovering the organic phase, and drying the organic phase.

18. The method according to claim 2, wherein the organic solvent of step b) is alcohol (C1-C6) and alkyl acetate.

19. A composition comprising the bacterial strain according to claim 1, or an extract thereof and at least one cosmetically or dermatologically acceptable excipient.
